(19) United States
(12) Patent Application Publication
(10) Pub. No.: US 2026/0317801 A1
(43) Pub. Date: Oct. 15, 2026

(54) ENZYMATICALLY PRODUCED GLUCOSYLATED MYCOSPORINES FOR DEVELOPING INNOVATIVE ANTI-UV PRODUCTS

(71) Applicants: Université de Pau et des pays de l’Adour, PAU (FR); Centre National de la Recherche Scientifique, PARIS (FR); Institut national de recherche pour l’agriculture, l’alimentation et l’environnement, PARIS (FR); Institut National des Sciences Appliquées de Toulouse, TOULOUSE (FR)

(72) Inventors: Susana FERNANDES, PAU (FR); Claire MOULIS, VIEILLEVIGNE (FR); Elodie BASCANS, TOULOUSE (FR); Marion CLAVERIE, BAUDREIX (FR); Etienne SEVERAC, PIBRAC (FR); Magali REMAUD-SIMEON, RAMONVILLE-SAINT-AGNE (FR)

(21) Appl. No.: 19/101,247

(22) PCT Filed: Aug. 3, 2023
(86) PCT No.: PCT/FR2023/051238

§ 371 Date: Feb. 4, 2025

(30) Foreign Application Priority Data
Aug. 4, 2022 (FR) FR2208098

(51) Int. Cl. C12P 19/44; A61K 8/60; A61K 8/73; A61K 31/7032; A61K 31/716; A61Q 17/04; C12N 9/10; C12P 19/18

(52) U.S. Cl. CPC C12P 19/44; A61K 8/602; A61K 8/73; A61K 31/7032; A61K 31/716; A61Q 17/04; C12N 9/1051; C12P 19/18; A61K 2800/522; C12Y 204/01005; C12Y 204/0114

(57) ABSTRACT

The subject of the present disclosure is that of preparing glucosylated mycosporines by means of an enzymatic cascade method using α-transglucosylases and sucrose, and use thereof in particular for their anti-UV properties. A method for producing a glucosylated mycosporine includes a step a) of glucosylating a mycosporine by the enzyme α-transglucosylase GS-D from sucrose to obtain a glucosylation intermediate which is a glucosylated mycosporine having an average degree of polymerization ranging from 1 to 2 glucosyl units.

### CROSS-REFERENCE TO RELATED APPLICATIONS

The present application is a filing under 35 U.S.C. 371 as the National Stage of International Application No. PCT/FR2023/051238 filed Aug. 3, 2023, entitled “ENZYMATICALLY PRODUCED GLUCOSYLATED MYCOSPORINES FOR DEVELOPING INNOVATIVE ANTI-UV PRODUCTS,” which claims priority to French Application No. 2208098 filed with the Intellectual Property Office of France on Aug. 4, 2022, both of which are incorporated herein by reference in their entirety for all purposes.

### TECHNICAL FIELD

The present invention relates to preparing glucosylated mycosporines by means of an enzymatic cascade method using α-transglucosylases and sucrose, and use thereof in particular for their anti-UV properties (e.g. UV absorption and protection properties, antioxidant properties).

The present invention is for example applicable in the field of ophthalmology, dermatology, cosmetology, materials and agriculture.

In the following description, the references between brackets [ ] refer to the list of references presented at the end of the text.

### STATE OF THE ART

Prevention and protection are essential to deal with the chronic and acute damage caused by UV radiation to human health, particularly to the skin and eyes. Reducing UV exposure and applying a sunscreen is thus strongly recommended to prevent skin diseases. Broad-spectrum inorganic sunscreens composed of titanium dioxide (TiO₂) and zinc oxide (ZnO), which act primarily by absorption but also by diffusion of radiation, are commonly used [1]. However, their generally opaque and white appearance is not accepted by all consumers, and the possible cytotoxicity of nanoparticle accumulation in the body has not yet been sufficiently studied [2]. Organic filters composed of synthetic molecules have been accused of having relatively limited efficacy in protecting the skin, of sometimes being allergenic, and of being endocrine disruptors [1,2]. Some are even suspected of breaking down into carcinogenic compounds, such as octocrylene [3], which was recently shown to break down over time into benzophenone. In addition, the lack of photostability of certain compounds could have a counter-productive effect by creating ROS (Reactive Oxygen Species) and increasing oxidative stress. Finally, from an environmental point of view, these two types of filter are accused of accumulating in coastal waters, causing endocrine changes in fish and coral bleaching phenomena [4,1]. Additionally, in sunscreens, chemical filters such as oxybenzone cause allergies and side effects in humans, and are known to damage coral reefs [4]. It is therefore necessary to develop new solutions based on natural resources and using environmentally-friendly methods.

Mycosporines and mycosporine-like amino acids (MAAs) (hereinafter both referred to generically as mycosporines) are small molecules of approximately 400 dalton (Da), organized around a cyclohexenone or cyclohexenimine ring. Due to their structure, these zwitterions have unique UV absorption capabilities, with an absorption range from 309 to 362 nm and an extinction coefficient from 21,800 cm⁻¹·M⁻¹ to 50,000 cm⁻¹·M⁻¹ [5]. They therefore cover both the UV-A and UV-B range. Mycosporines are also described as powerful antioxidants. They are secondary metabolites and their role as a natural sunscreen has been studied for years. Their potential is promising for several reasons: (i) they can act as UV filters and antioxidants, an effective combination for protecting the body against the multiple damage caused by photolesions, (ii) they are also being studied for their anti-cancer, anti-aging and anti-inflammatory properties, and (iii) they have great potential for promoting wound healing [1]. Their mechanism of action is as follows: in a photoexcited state, they undergo rapid internal conversion to the ground electronic state (S0) by releasing heat or else by undergoing reversible isomerization [1,6]. By virtue of this mechanism, over 90% of the energy received is released by heat transfer to the surrounding environment without generating ROS. No fluorescence emission has been particularly reported [7].

Mycosporines are present in a wide variety of organisms. UV radiation impacts terrestrial life, but it can also penetrate waters 0.5 to 47 meters deep, depending on the clarity of the water and the weather conditions [1]. Mycosporines have thus been identified in cyanobacteria, but also in heterotrophic bacteria and marine fungi, microalgae, macroalgae (mainly red and brown algae), phytoplankton (diatoms, dinoflagellates), corals, molluscs and fish [7,8]. They have also been identified in terrestrial organisms, such as lichens living in sometimes very extreme conditions [9]. This is the case of mycosporine-serinol MSer(OH), notably present in the species Lichina pygmaea, found in the mediolittoral zones of the coasts of Western Europe.

In metazoans such as fish, mycosporines can rapidly accumulate in the tissues of sun-exposed outer layers, such as for example the skin of the dorsal surface, as well as sensitive organs such as the digestive tract, reproductive organs, and also the eyes [1,5,7,8]. In the eye, they are particularly concentrated in the cornea and lens, thus protecting the retina from photooxidative damage caused by UV rays [10].

The presence of mycosporines in the ocular tissues of fish is a particularly interesting subject for bioinspiration, especially in the development of preventive and/or curative biomedical solutions in the ophthalmological field. However, the formulation of biomedical products from currently available mycosporines is complicated due to their small size which leads to their rapid elimination from mucous membranes (especially the eye).

Glucosylated mycosporines are already present in nature, essentially with a degree of polymerization (DP) of 1 to 2 glucosyl units (that is, xylose, galactose, glucosamine, etc.), exceptionally up to 8 glucosyl units of different kinds (that is, xylose, galactose, glucosamine, etc.) in the case of a mycosporine-glycine-like mycosporine [11]. However, the most widely described are those found in the exopolysaccharide matrix of terrestrial cyanobacteria of the genus Nostoc (Nostoc commune, Nostoc sphaericum) [12-14]. In these organisms, various mycosporines have been identified (shinorine, porphyra-334, palythine, mycosporine-glycine, mycosporine-glutamicol) linked to different glucosyl units (galactose, glucosamine, xylose, etc.). Although the role of these glucosylated mycosporines has not yet been fully discovered, they probably play a multifunctional role in protecting against UV radiation, but also against heat stress or desiccation. The value of sugars covalently bound to mycosporines has not been fully elucidated either, although it has been strongly suggested that this would improve interactions with the exopolysaccharide matrix of these cyanobacteria [13]. In addition to the enhanced stability and non-covalent attachment to the extracellular matrix of glycans, the high proportion of glucosylated mycosporines surely testifies to another useful role: Ishihara et al, thus identified that 86.5% of the total Nostoc sphaericum mycosporines were β-galactose-porphyra-334; 13.2% were hexose-shinorines and only 0.2% were free porphyra-334 [14].

### DESCRIPTION OF THE INVENTION

The Inventors hypothesized that it might be interesting to incorporate long glucosylated chains into mycosporines in order to see whether the resulting molecules would be stable enough and retain their anti-UV properties (photostability) to be usable.

The Inventors have therefore developed a new method capable of adding a long glucosyl chain to mycosporines, resulting in new environmentally-friendly, highly UV-protective products or biomaterials to replace current UV filters, capable of extending the retention time of mycosporines in organs such as the eyes. The nature of the carbohydrate chain (composed solely of glucosyl units) should reduce the risk of allergic reaction.

The resulting UV-protective biomolecules (glucosylated mycosporine) with extended retention times using exclusively natural precursors can be applied in cosmetic formulations, medical products (e.g. in ophthalmology), biomaterials and outdoor materials as alternatives to current active ingredients.

To do this, although it has never yet been studied in relation to mycosporines, enzymatic glucosylation has emerged as the most effective method. A family of enzymes has emerged as particularly interesting for this type of enzymatic catalysis. These are the α-transglucosylases of Glycoside Hydrolase family 70 (GH-70) [15], enzymes naturally found in lactic acid bacteria of the genera Leuconostoc, Lactobacillus, Streptococcus, Oenococcus or Weissela sp. [16]. These enzymes are of considerable industrial interest due to their catalytic mechanism, shown in FIG. 16. From sucrose, a very inexpensive substrate derived from renewable resources (sugar beet and sugar cane), these enzymes are capable of forming a variety of products based on the nature of the acceptor molecule. After forming a covalent intermediate (step 1 in FIG. 16), GH-70 can transfer the glucosyl unit to [17]:
water (step 3 in FIG. 16) to carry out a hydrolysis reaction, in which free glucose is formed (mostly at the start of the reaction for very good polymerases of the GH-70 family).
a glucose and progressively glucose oligosaccharides of increasing size (step 4 on FIG. 16), to achieve the synthesis of homopolymers of α-D-glucosyl units, called α-glucans. These represent a wide variety of polymers, ranging from 10³ to 10⁹ Da and composed of osidic bonds of different natures (α-1,2; α-1,3; α-1,4 and/or α-1,6). Among these α-glucans, one of the best known is dextran. It is a more or less linear polymer containing a majority of α-1,6-linked glucosyl units and has numerous applications in industry (food, cosmetics and pharmaceutical domains for example).
fructose (step 5 in FIG. 16), sucrose isomers such as leucrose are formed for example. These products are nevertheless in the minority.
an exogenous acceptor molecule added to the medium at the start of the reaction (glycone or aglycone), having at least one hydroxyl function (step 6 in FIG. 16); this catalytic action is referred to as the acceptor reaction.

The ability to transfer the glucosyl units to these exogenous molecules depends on several factors including the structure of the acceptor and that of the active site of the enzyme. An efficient acceptor will be able to redirect almost all the transfer of glucosyl units not to polymer formation but to its own glucosylation. To date, a wide variety of acceptors have already been tested, for example flavonoids such as quercetin and luteolin [18].

The aim of the present invention is therefore to produce glucosylated derivatives of mycosporines, in particular mycosporine-serinol (hereinafter referred to as MSer(OH)) derived in particular from the marine lichen Lichina pygmaea, by virtue of an enzymatic cascade method using α-transglucosylases of the Glycoside Hydrolase family 70 (GH-70) and sucrose. Naturally, these enzymes use sucrose derived from sugar beet or sugar cane to synthesize (polymerases) or decorate (branching sucrases) high molar mass polymers of glucosyl units (α-glucans). By way of comparison, some sucrose phosphorylases that also use sucrose as a donor of glucosyl units, as well as commercial CGTases belonging to the GH-13 family that use starch (another natural and abundant substrate) as a donor of glucosyl units have also been tested, but without success.

The enzymatic cascade method described herein (FIG. 1) has the advantage of not involving any chemical reaction; this avoids several steps of protection/deprotection of the hydroxyl functions of sugars which generally require the use of metal catalysts or strong acids. These latter are known to generate large quantities of waste to be processed and can lead to uncontrolled parasitic reactions.

Glucosylated mycosporines are produced in a totally aqueous medium without the addition of organic solvents.

Glucosylation can be carried out in a single step (co-synthesis or one-pot synthesis) or in two steps as follows:
1/ glucosylation of a mycosporine (e.g. mycosporine-serinol, MSer(OH)) by the GS-D enzyme (α-transglucosylase of the GH-70 family) from sucrose, resulting in glucosylation intermediates called mycosporine-glu (e.g. MSer(OH)-glu);
2/ elongation of mycosporine-glu glucosylation intermediates (e.g. MSer(OH)-glu) by other α-transglucosylases of the GH-70 family from sucrose, resulting in final glucosylation products called mycosporine polym (e.g. MSer(OH)-polym).

This second step in the enzymatic cascade allows the “custom” synthesis of mycosporine covalently linked to glucosylated chains of varying lengths and osidic bonds (α-1,2; α-1,3; α-1,4 and/or α-1,6), depending on the enzymes and reaction conditions implemented. Mycosporine-glu intermediates (e.g. MSer(OH)-glu) can carry up to 4 glucosyl units with an average degree of polymerization (DP) predominantly centered between 1 and 2 glucosyl units, while mycosporine-polym end products (e.g. MSer(OH)-polym) can have between 2 and 100,000 glucosyl units, with an average degree of polymerization (DP) centered between 3 and 1,000,000, between 3 and 800,000, between 3 and 500,000, between 3 and 24,000, between 3 and 60, or between 3 and 41 glucosyl units, depending on the enzymes used.

As mycosporines are considered natural sunscreens, the mycosporine derivatives developed by the method of the present invention composed of mycosporines having a long glucosylated chain could easily be used for the prevention and protection of cells (dermal and ocular) and have cosmetic, medical and/or pharmaceutical applications. In particular, applications in ophthalmological products for eye protection (e.g. cataract prevention) or for the protection of people with genetic and hereditary abnormalities such as albinism and xeroderma pigmentosum can be envisaged. Another application would be to incorporate these glucosylated mycosporines into biosourced, biodegradable sunscreens without environmental consequences such as coral bleaching. Finally, these glucosylated mycosporines can also be used to protect materials used outdoors [19].

The object of the present invention is therefore a method for producing a glucosylated mycosporine, said method comprising a step a) of glucosylating a mycosporine by the enzyme α-transglucosylase GS-D from sucrose to obtain a glucosylation intermediate being a glucosylated mycosporine having a degree of polymerization ranging from 1 to 4 glucosyl units, preferably an average degree of polymerization of 1 to 2 glucosyl units.

According to a particular embodiment of the present invention, the method may further comprise a step b) of elongating the carbohydrate portion of the glucosylation intermediate obtained in step a) from sucrose by at least one α-transglucosylase other than α-transglucosylase GS-D to obtain a glucosylated mycosporine having an average degree of polymerization ranging from 3 to 1,000,000 glucosyl units, from 3 to 800,000 glucosyl units, from 3 to 500,000 glucosyl units, from 3 to 24,000 glucosyl units (for a very good polymerase such as for example the DSR-OK Δ1 dextransucrase enzyme), from 3 to 60 glucosyl units, from 3 to 41 glucosyl units; preferably from 9 to 1,000,000 glucosyl units, from 9 to 800,000 glucosyl units, from 9 to 500,000 glucosyl units, from 9 to 24,000 glucosyl units, from 9 to 60 glucosyl units, or from 9 to 41 glucosyl units.

According to a particular embodiment of the method of the present invention, steps a) and b) can be carried out concomitantly, namely that said method comprises the glucosylation of a mycosporine and the elongation of its carbohydrate portion by the enzyme α-transglucosylase GS-D and at least one other α-transglucosylase different from α-transglucosylase GS-D from sucrose, to obtain a glucosylated mycosporine having an average degree of polymerization ranging from 3 to 1,000,000 glucosyl units, from 3 to 800,000 glucosyl units, from 3 to 500,000 glucosyl units, from 3 to 24,000 glucosyl units (for a very good polymerase such as for example the DSR-OK Δ1 dextransucrase enzyme), from 3 to 60 glucosyl units, from 3 to 41 glucosyl units; preferably from 9 to 1,000,000 glucosyl units, from 9 to 800,000 glucosyl units, from 9 to 500,000 glucosyl units, from 9 to 24,000 glucosyl units, from 9 to 60 glucosyl units, or from 9 to 41 glucosyl units.

For the purposes of this invention, “average degree of polymerization (DP)” means the degree of polymerization determined at the apex of the peaks obtained by high-performance size-exclusion chromatography analysis coupled to a UV detector and column calibration using commercial dextrans with a molar mass of between 342 g/mol and 70,000 g/mol.

According to a particular embodiment of the method of the present invention, said at least one α-transglucosylase of step b) is selected from the group consisting of: glucansucrase GS-A, GS-B, GS-C, GS-F Δ1, and GS-FS; dextransucrase DSR-M Δ1, DSR-M Δ2 W624A, DSR-G Δ1, DSR-S vardel Δ4N, and DSR-OK Δ1; alternansucrase ASR Δ1.

According to a particular embodiment of the method of the present invention, the mycosporine of step a) is selected from the group consisting of: pure or purified mycosporine, extract of lichen Lichina pygmaea enriched in mycosporine from 0 to 50% by weight of extract, preferably from 5 to 35% by weight of extract, preferentially from 15 to 25% by weight of extract, most preferentially from 10% by weight of extract. Preferably, the mycosporine used in step a) is mycosporine-serinol (MSer(OH)). This latter can be isolated from extracts of cyanolichens such as Lichina pygmaea and Lichina confinis, various species of Peltigera, Stereum hirsutum, Alternaria chrysanthemi, Ascochyta pisi, Ophiobolus graminis, Pleospora herbarum or else Pyronema omphalodes. It can also be produced from its precursor (gadusol) by synthetic biology.

Another object of the present invention is a glucosylation intermediate obtained by the method of the present invention, said glucosylation intermediate being a glucosylated mycosporine having an average degree of polymerization ranging from 1 to 4 glucosyl units (alpha type bonds), preferably from 1 to 2 glucosyl units (alpha type bonds).

Another object of the present invention is a glucosylated mycosporine obtained by the method of the present invention, said glucosylated mycosporine having an average degree of polymerization ranging from 3 to 100,000 glucosyl units (alpha type bonds), from 3 to 800,000 glucosyl units (alpha type bonds), from 3 to 500,000 glucosyl units (alpha type bonds), from 3 to 24,000 glucosyl units (alpha type bonds), from 3 to 60 glucosyl units (alpha type bonds), or from 3 to 41 glucosyl units (alpha type bonds); preferably from 9 to 1,000,000 glucosyl units (alpha type bonds), from 9 to 800,000 glucosyl units (alpha type bonds), from 9 to 500,000 glucosyl units (alpha type bonds), from 9 to 24,000 glucosyl units (alpha type bonds), from 9 to 60 glucosyl units (alpha type bonds), or from 9 to 41 glucosyl units (alpha type bonds).

A further object of the present invention is the cosmetic use of a glucosylated mycosporine according to the present invention, as an anti-UV agent, to (i) protect the skin from UV rays, (ii) combat photoaging.

A further object of the present invention is a cosmetic composition comprising as active ingredient a glucosylated mycosporine according to the present invention.

A further object of the present invention is the cosmetic use of a cosmetic composition according to the present invention, to (i) protect the skin from UV rays, (ii) combat photoaging.

A further object of the present invention is a glucosylated mycosporine according to the present invention for use as a medicine, for use in the prevention or treatment of, for example, cataract, skin diseases associated with UV sensitivity such as xeroderma pigmentosum, hereditary genetic diseases such as albinism, or else skin cancer.

In conclusion, the invention implements green processes and mobilizes natural resources, namely mycosporines, notably derived from a lichen growing on the seashore, and sucrose. This latter resource, which serves as a substrate for GH-70 family enzymes, is available in large quantities and at low cost. Enzymatic reactions are processes under mild conditions and produce no (toxic) waste. Some glucosylated chains have dextran signatures, an α-glucan widely used in the cosmetics and biomedical fields, as it is very well tolerated by the body. As the dextran chain of MSer(OH)-polym products can be “custom” modulated (that is, in terms of osidic bonds, carbohydrate chain size), it can easily be adapted to the type of application targeted. In nature, in the mucus and lens of fish for example, mycosporines are bound to proteins [7,20,21]. Within the scope of the present invention, this phenomenon has been imitated by producing MSer(OH)-polym mycosporine derivatives with an elongated carbohydrate chain. Compared with free mycosporine molecules, glucosylated mycosporines obtained by the method of the present invention have better properties, particularly photostability.

Compared with commercial UV protection molecules such as for example oxybenzone (a UVA and UVB blocker) which causes bleaching of coral reefs [4], glucosylated mycosporines do not cause such damage.

### BRIEF DESCRIPTION OF THE FIGURES

FIG. 1 shows the principle of the enzymatic cascade for the glucosylation of mycosporines from sucrose and involving α-transglucosylases of the GH-70 family: an initial glucosylation step carried out by the GS-D enzyme, followed by elongation of the glucosidic chain by different enzymes of the family, allowing both control of the size and structure of this chain (different types of α-osidic bonds depending on the enzyme selected).

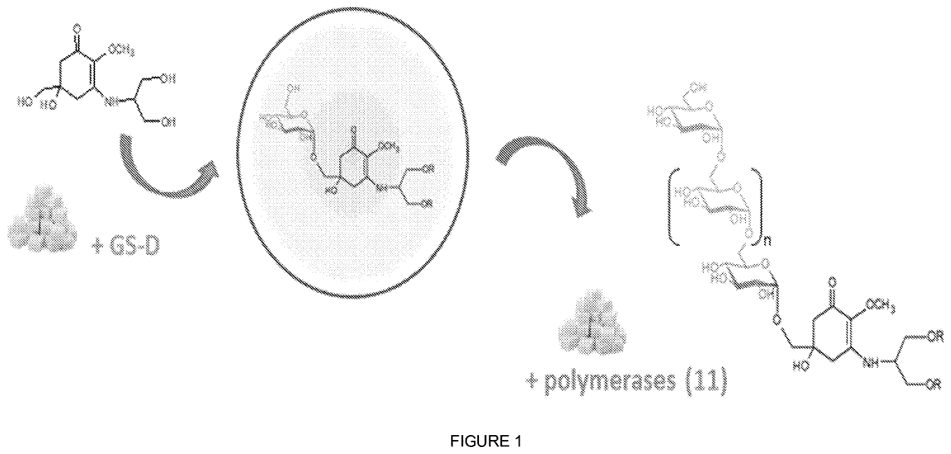

FIG. 2 depicts UV chromatograms (λ=310 nm) of the α-transglucosylase enzymes of the GH-70 family screened for MSer(OH) glucosylation. Only the GS-D enzyme can efficiently glucosylate MSer(OH).

FIG. 3 depicts UV chromatograms (λ=310 nm) of the commercial CGTase and sucrose phosphorylase enzymes screened for MSer(OH) glucosylation.

FIG. 4 depicts UV chromatograms (λ=310 nm) of the different glucosylation products of 5 mM of MSer(OH) by GS-D, called MSer(OH)-glu. Numbering 1-8 identifies the different MSer(OH)-glu glucosylation products obtained at the end of the first step of the enzymatic cascade.

FIGS. 5A-5D show (FIG. 5A) the chemical structure of MSer(OH) with carbon numbering and illustration of possible glucosylation positions. Hexagons represent glucosyl units bound to MSer(OH) (FIG. 5B) which are example structures of DP2 produced by glucosylation of MSer(OH) by the GS-D enzyme (FIG. 5C) which are example structures of DP3 produced by glucosylation of MSer(OH) by the GS-D enzyme (FIG. 5D) which are example structures of products from the enzymatic cascade (glucosylation+elongation).

FIG. 6 shows 1H NMR (spectrum A) and HMBC (spectrum B) spectra confirming the glucosylation of MSer(OH) by GS-D into a glucosylation intermediate (MSer(OH)-glu) with an average degree of polymerization ranging from 1 to 2 glucosyl units.

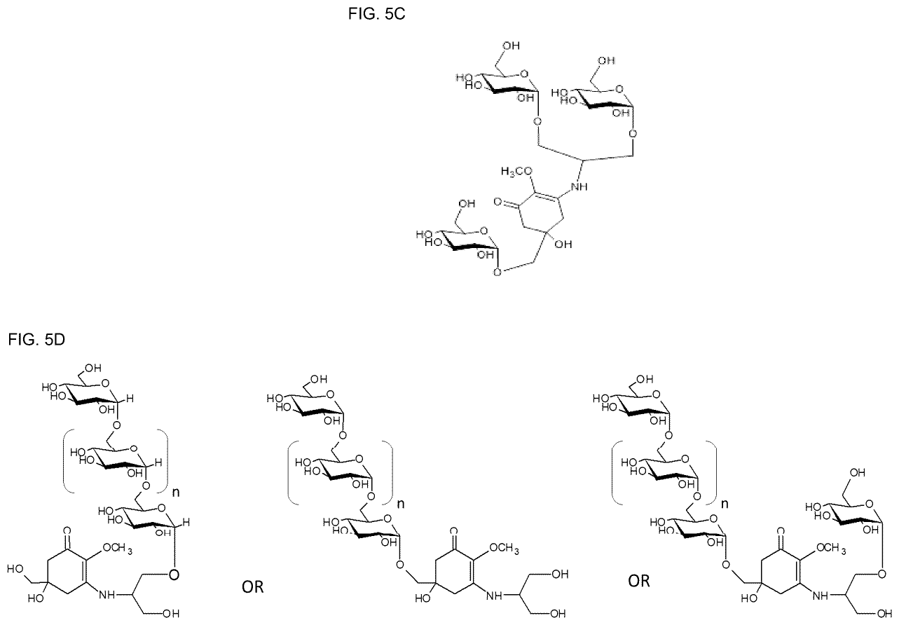

FIG. 7 shows the comparison of UV chromatograms (λ=310 nm) of glucosylation reactions of MSer(OH) by GS-D, at 5 and 50 mM of MSer(OH).

FIG. 8 shows UV chromatograms (λ=310 nm) of GS-D enzymatic cascade polymerizations followed by a polymerase (that is, another α-transglucosylase of the GH-70 family). The enzymes used in the second step are indicated on the chromatograms.

FIG. 9 depicts the UV chromatograms (λ=310 nm) of 6 of the 11 different reactions, separated by high-performance size-exclusion chromatography (HPSEC). The chromatogram labeled DEX 10,000 Da is that of a commercial dextran with an average molar mass of 10,000 g/mol. Asterisks identify the majority population(s) for each reaction. Peaks DP1 and DP2 correspond respectively to Peak 1 and Peak 2 in FIG. 4.

FIG. 10 depicts the UV chromatograms (λ=310 nm) of 3 of the 11 different reactions, separated by high-performance size-exclusion chromatography (HPSEC). The chromatogram labeled DEX 70,000 Da is that of a commercial dextran with an average molar mass of 70,000 g/mol.

FIG. 11 shows the comparison of HPLC analyses on a Synergi™ Fusion-RP C18 column coupled to a UV detector (λ=310 nm) of polymerization reactions of MSer(OH)-glu products by the DSR-M Δ2 enzyme and its DSR-M Δ2 W624A mutant.

FIG. 12 shows the absorbance scans of MSer(OH) and the MSer(OH)-glu and MSer(OH)-polym products for wavelengths ranging from 200 nm to 800 nm (area of interest shown: 200 to 400 nm).

FIG. 13 shows the comparison of UV chromatograms (λ=310 nm) of GS-D glucosylation reactions on purified MSer(OH) present up to 10% in a crude extract of lichen Lichina pygmaea, in reactions carried out with 6.37 mM of MSer(OH) and 292 mM of sucrose.

FIG. 14 shows the comparison of UV chromatograms (λ=310 nm) of the glucosylation reactions of GS-D and DSR-M Δ1 on purified MSer(OH) and that present in the crude extract derived from the lichen Lichina pygmaea.

FIG. 15 shows the chromatograms of co-synthesis polymerizations. MSer(OH)-glu products are less efficiently consumed by polymerases, and fewer MSer(OH)-polymers are produced.

FIG. 16 shows a schematic diagram of the different catalytic mechanisms of GH-70.

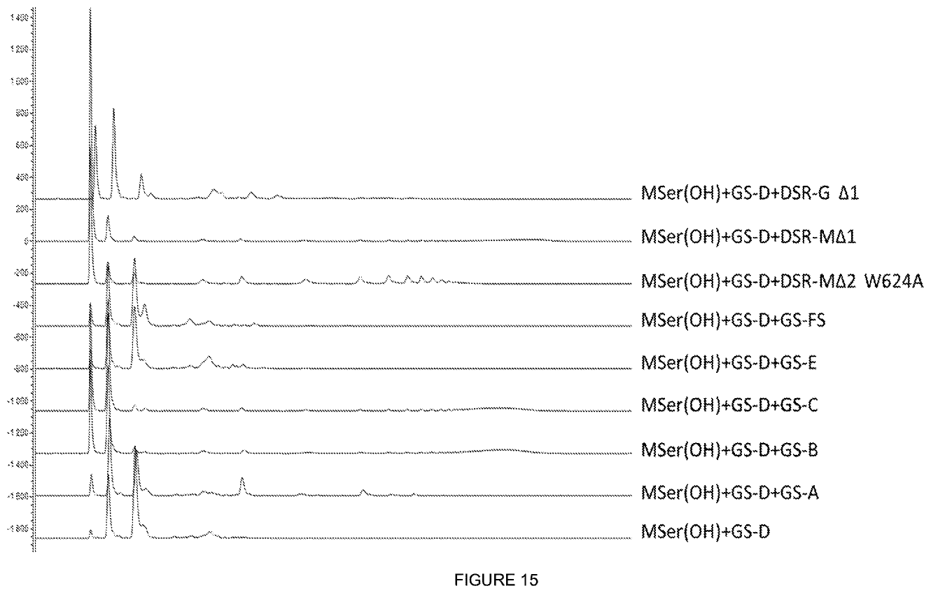

FIG. 17 shows the quantum yield of MSer(OH) and the various MSer(OH)-glu and Mser(OH)-polym glucosylation products obtained by the method of the invention.

FIG. 18 shows the TEAC score of MSer(OH) and the products of the enzymatic cascade of the method of the invention, with and without MSer(OH).

### EXAMPLES

### Example 1: Example Implementation of the Method of the Invention

### First Screening

A collection of α-transglucosylases of the GH70 and GH13 families was screened for their ability to glucosylate the mycosporine-serinol (MSer(OH)) molecule from sucrose or optionally starch. Initially, the work was carried out using pure MSer(OH) obtained from an extract of Lichina pygmaea. The enzymes listed in Table 1 below have all been produced in recombinant form in Escherichia coli, with the exception of CGTases and sucrose phosphorylase (enzymes no. 22, 23 and 24), which are commercial formulations.

TABLE 1

| | | Catalysis | | | | |
|---|---|---|---|---|---|---|
| | | on natural | 1^{st} | 2^{nd} | Original | GenBank |
| No. | Enzyme | substrate | screening | screening | organism | accession no. |
| 1 | GS-A | P | x | x | Lb. kunkeei MP2 | ALJ31412.1 |
| 2 | GS-B | P | x | x | Lb. animalis | KRM57462.1 |
| | | | | | DSM 20602 | |
| 3 | GS-C | P | x | x | Lb. apodemi | WP_056957205.1 |
| 4 | GS-D | N.D | x | | Lb. animalis | KRM57463.1 |
| | | | | | DSM 20602 | |
| 5 | GS-E | P | x | | Lb. capillatus | KRL03580.1 |
| | | | | | DSM 19910 | |
| 6 | GS-F Δ1 | P | x | x | L. fallax | WP_010006777.1 |
| | | | | | KCTC 3537 | |
| 7 | GS-FS | P | x | x | S. salivarius | ALR80278.1 |
| 8 | DSR-M Δ1 | P | x | x | L. citreum NRRL | CDX66895.1 |
| | | | | | B-1299 | (sequence |
| | | | | | | between amino |
| | | | | | | acids 42 to 1433) |
| 9 | DSR-M Δ2 | P | | x | L. citreum NRRL | CDX66895.1 |
| | W624A | | | | B-1299 | (amino acids 42 to |
| | | | | | | 1433 + W624A |
| | | | | | | mutation) |
| 10 | DSR-M Δ2 | P | | | L. citreum NRRL | CDX66895.1 |
| | | | | | B-1299 | (amino acids 164 to |
| | | | | | | 1433) |
| 11 | DSR-G Δ1 | Bif | x | x | Lb. kunkeei | KRK22577.1 |
| | | | | | DSM 12361 | (sequence |
| | | | | | | between amino |
| | | | | | | acids 50 and 2264) |
| 12 | ASR Δ1 | P | x | x | L. mesenteroides | CAB65910.2 |
| | | | | | NRRL B-1355 | (sequence |
| | | | | | | between amino |
| | | | | | | acids 1 and 1425) |
| 13 | BRS-A | B | x | | L. citreum NRRL | CDX66896.1 |
| | | | | | B-1299 | |
| 14 | BRS-B Δ1 | B | x | | L. citreum NRRL | CDX65123.1 |
| | | | | | B-742 | (sequence |
| | | | | | | including amino |
| | | | | | | acids 39 to 1313) |
| 15 | BRS-C | B | x | | L. fallax | ZP_08312597.1 |
| | | | | | KCTC 3537 | |
| 16 | BRS-D | B | x | | Lb. kunkeei | WP_051592287 |
| | | | | | EFB6 | |
| 17 | BRS-E | B | x | | L. mesenteroides | AHF19404.1 |
| | | | | | KFRI-MG | |
| 18 | BRS-F | B | x | | F. tropaeoli | GAP05007.1 |
| 19 | GBD-CD2 WT | B | x | | L. citreum NRRL | CDX66820.1 |
| | | | | | B-1299 | |
| 20 | GBD-CD2 | B | x | | L. citreum NRRL | CDX66820.1 (19 |
| | W21355- | | | | B-1299 | with W21355 and |
| | F2136L | | | | | F2136L mutations) |
| 21 | GBD-CD2 | B | x | | L. citreum NRRL | CDX66820.1 (19 |
| | F2136G | | | | B-1299 | with F2136G |
| | | | | | | mutation) |
| 22 | DSR-S vardel | P | | x | L. mesenteroides | I09598 (sequence |
| | Δ4N | | | | NRRL B-512F | including amino |
| | | | | | | acids 152 to 1450) |
| 23 | DSR-OK Δ1 | P | | x | O. kitaharae | EHN59583.1 |
| | | | | | DSM 17330 | (sequence |
| | | | | | | including amino |
| | | | | | | acids 84 to 1484) |
| 24 | Sucrose | S-Php | x | x | Bb. adolescentis | Commercial |
| | phosphorylase | | | | | enzyme |
| | 13A | | | | | |
| 25 | “Amano” | GT | x | x | B. macerans | Commercial |
| | CGTase | | | | | enzyme |
| 26 | Toruzyme 3.0 | GT | x | x | B. licheniformis | Commercial |
| | L CGTase | | | | | enzyme |

L. == Leuconostoc; Lb. = Lactobacillus; Bb. = Bifidobacterium; P = Polymerase; B = Branching sucrase; Bif = bifunctional; GT = α-glucanotransferases; S-Php = sucrose phosphorolysis; N.D = not determined.

The enzymes were produced from E. coli BL21 Star (DE3) cells transformed with the plasmid containing the target enzyme gene (Table 2). 300 μL of the transformation mixture was used to inoculate a 30 mL volume of LB (Lysogeny Both), supplemented with 100 μg/mL ampicillin. The medium was incubated overnight at 37° C. to prepare a preculture. 1 L cultures in modified ZYM5052 medium [22], whose characteristics are presented in Table 2, were inoculated at an initial OD (λ=600 nm) of 0.05 from the previous day's preculture, then incubated for 26 hours at 21° C. and 150 rpm.

At the end of fermentation, the culture media were centrifuged (15 minutes, 800 g, 8° C.) and the pellets concentrated to an OD (λ=600 nm) of 80 in 50 mM sodium acetate buffer pH=5.75. The cells were then broken by ultrasound according to the following protocol: 5 cycles of 20 seconds at 30% of the maximum power of the probe, cold, separated by 4 minutes of rest in ice. Sonication supernatants containing soluble enzymes of interest were then recovered after 30 minutes of centrifugation (15,000 g, 6° C.) and stored at 4° C.

TABLE 2

| | | | Culture medium conditions | | |
|---|---|---|---|---|---|
| No. | Enzyme | Plasmid | Gly | Glu | Inductor |
| 1 | GS-A | pET23a | 0.5% | 0.05% | 0.2% α-Lac |
| 2 | GS-B | pET23a | 0.5% | 0.05% | 0.2% α-Lac |
| 3 | GS-C | pET23a | 0.5% | 0.05% | 0.2% α-Lac |
| 4 | GS-D | pET23a | 0.5% | 0.05% | 0.2% α-Lac |
| 5 | GS-E | pET23a | 0.5% | 0.05% | 0.2% α-Lac |
| 6 | GS-F Δ1 | pET53 | 1% | 0% | 1% α-Lac |
| 7 | GS-FS | pET23a | 0.5% | 0.05% | 0.2% α-Lac |
| 8 | DSR-M Δ1 | pET55 | 1% | 0% | 0.1% α-Lac |
| 9 | DSR-M Δ2 | pET55 | 1% | 0% | 0.1% α-Lac |
| | W624A | | | | |
| 10 | DSR-M Δ2 | pET55 | 1% | 0% | 0.1% α-Lac |
| 11 | DSR-G Δ1 | pET55 | 1% | 0% | 0.1% α-Lac |
| 12 | ASR Δ1 | pBAD49 | 0.5% | 0.05% | 0.01% L-Ara |
| 13 | BRS-A | pBAD49 | 0.5% | 0.05% | 0.01% L-Ara |
| 14 | BRS-B Δ1 | pET55 | 1% | 0% | 0.1% α-Lac |
| 15 | BRS-C | pET55 | 1% | 0% | 0.1% α-Lac |
| 16 | BRS-D Δ1 | pET53 | 1% | 0% | 1% α-Lac |
| 17 | BRS-E Δ1 | pET60 | 1% | 0% | 0.1% α-Lac |
| 18 | BRS-F | pET23a | 0.5% | 0.05% | 0.2% α-Lac |
| 19 | GBD-CD2 | pET53 | 1% | 0% | 1% α-Lac |
| | WT | | | | |
| 20 | GBD-CD2 | pET53 | 1% | 0% | 1% α-Lac |
| | W21355- | | | | |
| | F2136L | | | | |
| 21 | GBD-CD2 | pET53 | 1% | 0% | 1% α-Lac |
| | F2136G | | | | |
| 22 | DSR-S vardel | pBAD49 | 0.5% | 0.05% | 0.01% L-Ara |
| | Δ4N | | | | |
| 23 | DSR-OK Δ1 | pET53 | 1% | 0% | 1% α-Lac |

Gly = Glycerol;
Glu = Glucose;
α-Lac = α-Lactose;
L-Ara = L-arabinose

Enzyme activity was determined by measuring the initial rate of production of reducing sugars using the dinitrosalicylic acid (DNS) method [23]. One enzyme unit represented the amount of enzyme that releases one μmol of fructose per minute, at 30° C., from 100 g/L of sucrose in 50 mM of sodium acetate buffer, pH=5.75. During kinetics, 100 μL of reaction medium was removed and the reaction stopped by adding an equivalent volume of DNS. The samples were then heated for 5 minutes at 95° C., cooled in ice, diluted by half in water, and the absorbance read off at λ=540 nm. A standard range of 0 to 2 g/L 10 of fructose was used to establish a link between absorbance value and reducing sugar concentration.

The first screening, which involved all the enzymes in Table 1, was carried out in a 25 μL volume containing 5 mM of MSer(OH), 292 mM of sucrose and 50 mM of sodium acetate buffer at pH=5.75. The reaction was incubated at 30° C. with 500 rpm stirring. After 24 hours, the reaction mix was incubated at 70° C. for 10 minutes to inactivate the enzymes. For analysis, the reaction media were diluted 1:10 in water and centrifuged for 5 minutes at 2000 g.

Separation of the various products (MSer(OH)-glu) was carried out in reverse phase using a 250 mm×2 mm C18 Synergi™ Fusion-RP column (porosity: 80 Å, particle size: 4 μm, Phenomenex, USA). This column was maintained at 30° C. on a Thermo Ultimate 3000 HPLC system equipped with a UV detector (Thermo Ultimate 3000) at λ=310 nm and a Corona Véo CAD (Charged Aerosol Detector) detector (nebulization temperature: 50° C.). The mobile phase was composed of a mixture of ultrapure water (solvent A)/LC-MS grade acetonitrile (solvent B) at a flow rate of 0.2 mL/min. Separation was achieved in 75 minutes using a linear gradient, as defined in Table 3.

TABLE 3

| Time (min) | % A | % B |
|---|---|---|
| 0 | 100 | 0 |
| 12 | 100 | 0 |
| 70 | 95 | 5 |
| 75 | 95 | 5 |

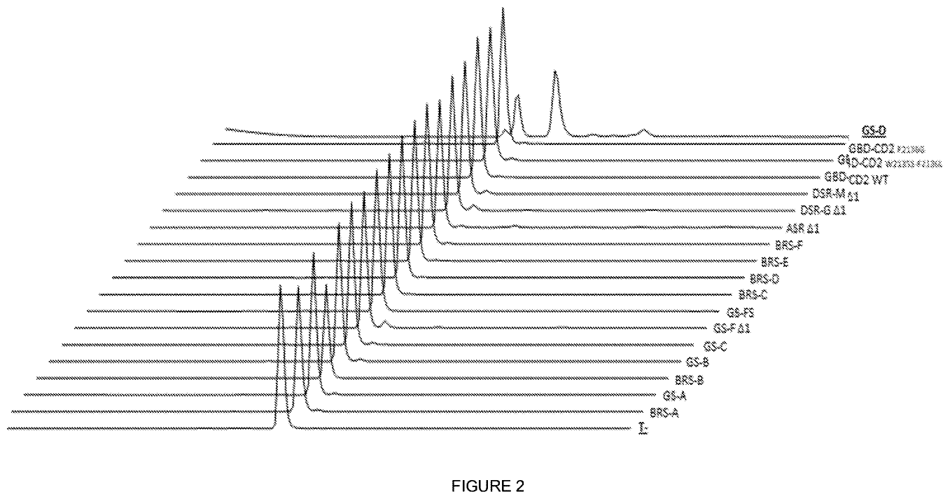

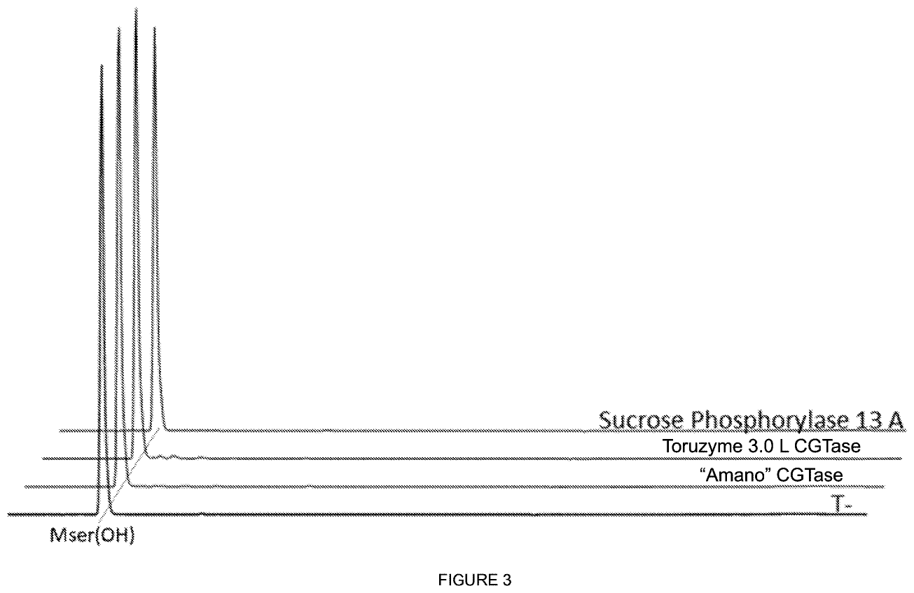

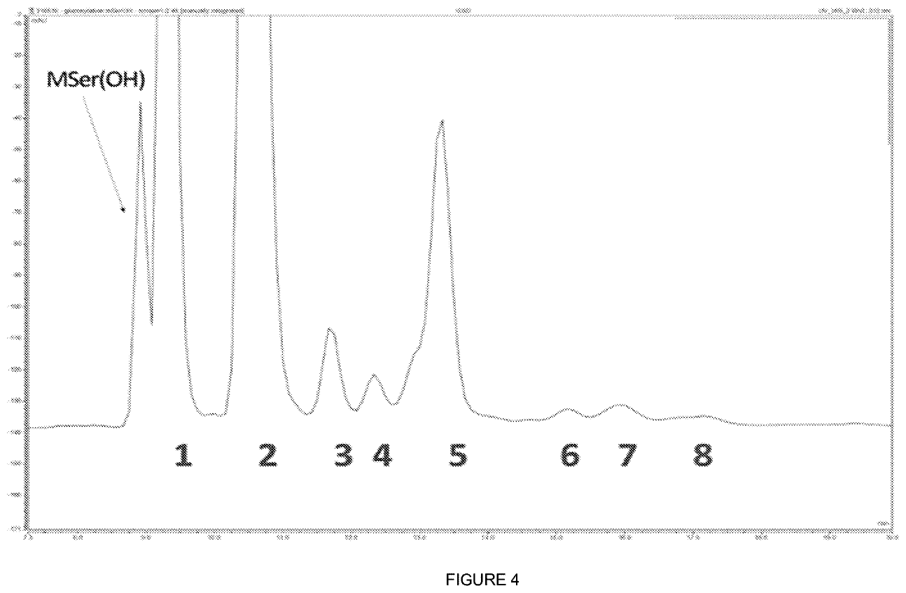

During this step, the GS-D enzyme proved to be by far the most efficient (FIGS. 2 and 3), converting 95% of MSer(OH) into 8 different glucosylated products, whereas for all the other enzymes tested, the conversion rate was between 0% and 6.5%. Representing 72.3% alone of the total area of MSer(OH)-glu products, the main peaks are the peaks numbered 1 and 2 in FIG. 4. Their analysis by liquid chromatography coupled to a mass detector (LC-MS) showed that they had a degree of polymerization (DP) of 1 and 2 glucosyl units respectively. Minority peaks (products 3 to 8 in FIG. 4) are thought to have a degree of polymerization (DP) of 3 to 4 glucosyl units.

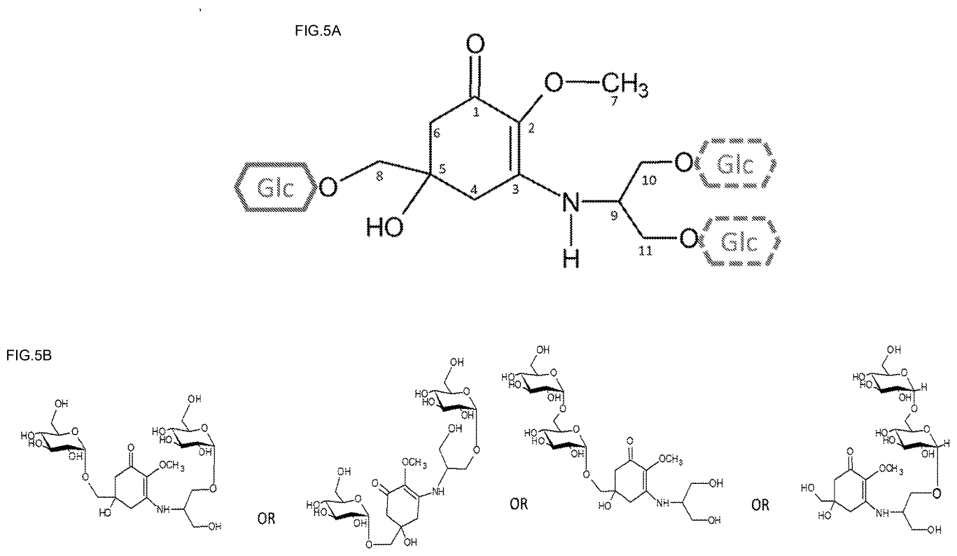

To identify the position of glucosyl units on MSer(OH)-glu, Nuclear Magnetic Resonance (NMR) analyses were carried out. ¹H, ¹³C, JMod, HSQC and HMBC spectra were recorded on Bruker Avance 500 MHz equipment at 298K with a 5 mm BBI H-BB-D probe with Z-gradient. Data were acquired and processed using TopSpin 3.5 software. All of these analyses showed that glucosylation took place on the hydroxyls carried by the C8 carbon and the C10/C11 equivalent carbons of MSer(OH) (FIG. 5A). FIGS. 5B-D show the structure of the products obtained by the method of the invention, and that the number of substitutions by glucosyl units can vary from 1 to 2, possibly 3, and the variety of glucosylated products that the culture medium can comprise.

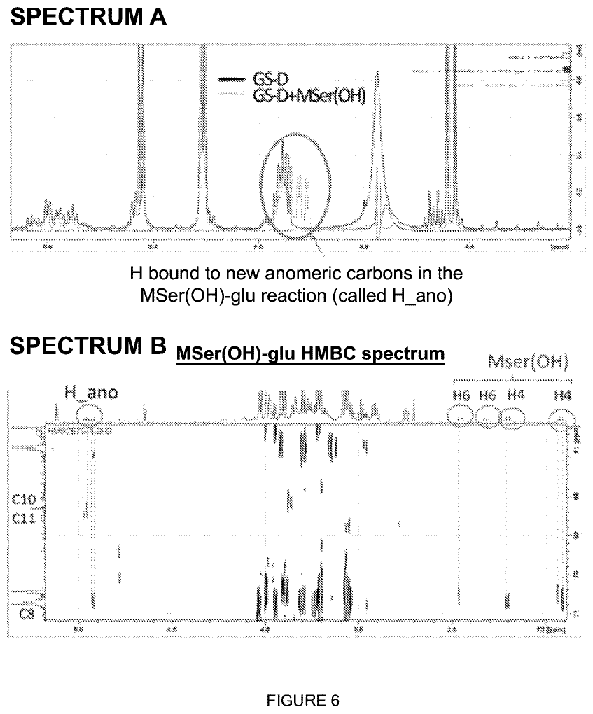

In order to assess the possibility of industrial application, the glucosylation reaction of MSer(OH) by GS-D was tested on a MSer(OH) concentration of 50 mM, that is, a 10-fold higher concentration than during screening. The protocol followed was identical to that described previously. In particular, with the sucrose concentration maintained at 292 mM, the sucrose: MSer(OH) molar ratio fell from 60:1 to 6:1. The comparison of the chromatograms for these two reactions is depicted in FIG. 7. The conversion rate of MSer(OH) into MSer(OH)-glu just changed from 95% with 5 mM of MSer(OH) to 90% with 50 mM of MSer(OH). This new satisfactory conversion rate showed that it was possible to increase the quantities of glucosylated product in a batch mode reaction. It was observed that the relative proportion between peaks 1 and 2 changed slightly, with the product corresponding to diglucosylated MSer(OH) (peak 2) being favored when the initial concentration is 5 mM (that is, when the sucrose: MSer(OH) ratio is highest).

### Second Screening

Following these initial results, the extension of MSer(OH)-glu glucosylated products from sucrose by certain polymerases of the GH-70 family was the subject of a second screening (see Table 1). An MSer(OH)-polym product was obtained whose glucoside portion was significantly lengthened with respect to that obtained by GS-D.

MSer(OH)-glu was produced under conditions similar to those described in the first screening but in larger quantities. The reaction was carried out in a 50 μL volume containing 25 mM of pure MSer(OH), 292 mM of sucrose, 1 μ·mL⁻¹ of GS-D and 50 mM of sodium acetate buffer at pH=5.75. This was incubated at 30° C. with 500 rpm stirring. After 24 hours, the reaction medium was incubated at 70° C. for 10 minutes to inactivate the enzyme.

The elongation of MSer(OH)-glu intermediates by other enzymes of the same family was carried out without prior purification of MSer(OH)-glu: 5 μL of reaction medium from step 1 was incorporated into a new 25 μL reaction such that the final molar concentration of MSer(OH)-glu was equivalent to 5 mM of free MSer(OH). To this were added the equivalent of 292 mM of sucrose and 1 U·mL⁻¹ of enzyme selected for the second step of the enzymatic cascade (see Table 1). This new medium also contained 50 mM of sodium acetate buffer at pH=5.75. Reactions were incubated at 30° C. with 500 rpm stirring for 24 hours, then stopped by heating at 70° C. for 10 minutes.

Separation of the different products (MSer(OH)-polym) was carried out in the same way as for MSer(OH)-glu in the first screening.

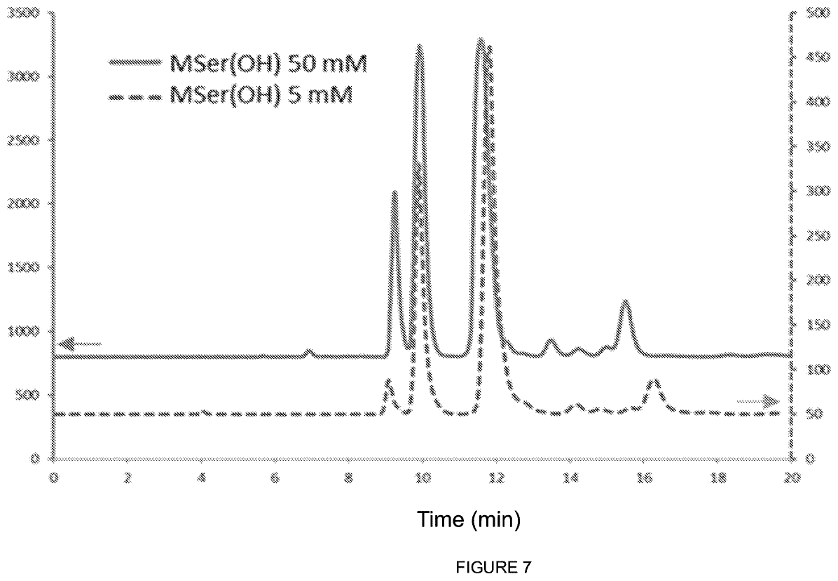

As shown in FIG. 8, the 11 polymerases tested were able to extend MSer(OH)-glu intermediates with glucosyl unit chains of varying size and nature depending on the binding specificity of the enzyme tested. Across all reactions, at least 67.6% and up to 97.7% of MSer(OH)-glu intermediates were elongated (Table 4). Peaks 1 and 2 of the MSer(OH)-glu intermediates (species in the overwhelming majority in the medium) were mainly consumed by polymerases, with the conversion percentage of each of these two peaks varying according to the specificity of the enzyme used (Table 4). For most enzymatic cascades, peak 2 (diglucosylated MSer(OH)) is consumed preferentially and sometimes almost entirely.

The average mass of elongated MSer(OH)-glu carbohydrate chains (that is, MSer(OH)-polym) was analyzed by size-exclusion chromatography (HPSEC) on a Shodex 802.5 column. Elution was carried out isocratically at a water flow rate of 0.3 mL/min and a temperature of 70° C. In the case of the elongation of MSer(OH)-glu intermediates by the DSR-OK Δ1 and DSR-S vardel Δ4N enzymes which form much larger natural polymers, HPSEC analysis was carried out by combining a Shodex 805 column and a Shodex 802.5 column in series. Elution was carried out with a mixture of 450 mM NaNO₃, 1% v/v ethylene glycol.

Separation calibration was based on a standard range of commercial dextrans (polymers of α-1,6-linked glucosyl units) of increasing size between 342 and 70,000 Da. The average masses MMₐᵥ of the products were determined at the apex of the elution peaks. The average degree of polymerization (DP av) visible under UV light was estimated by virtue of the following relationship: DPₐᵥ=MMₐᵥ/162.

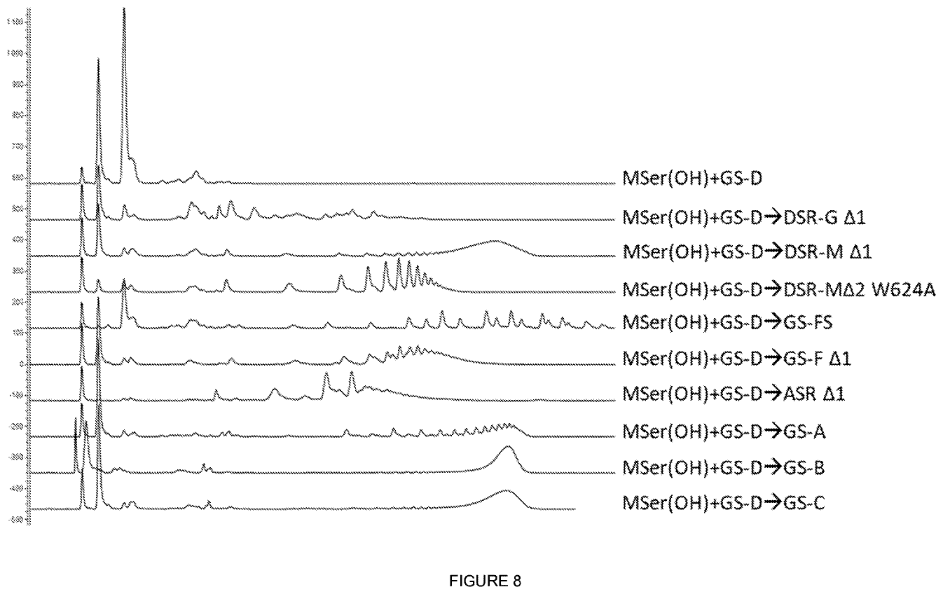

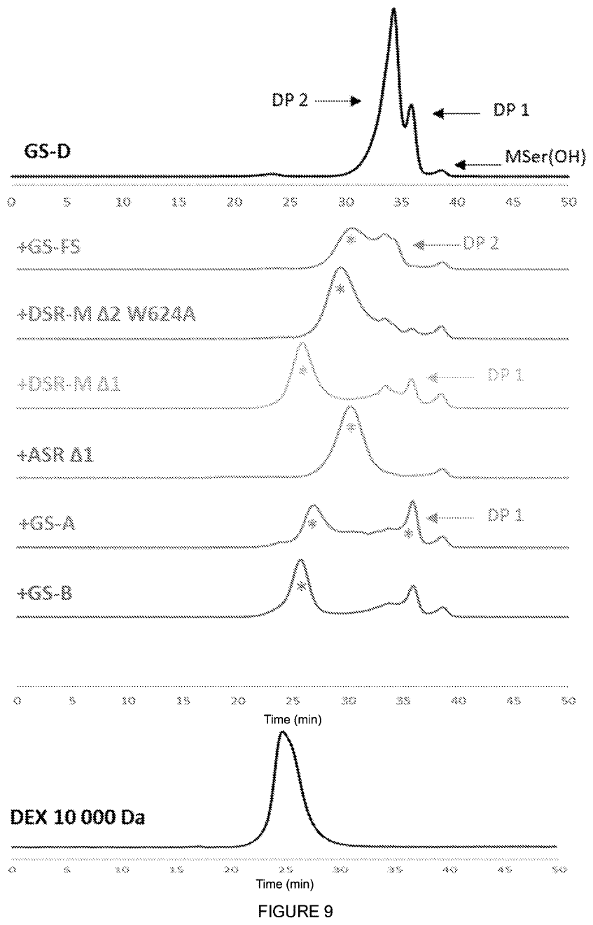

Depending on the polymerase used in this second step, glucosyl unit chains ranging from 3 to 41 units on average were identified (Table 4, FIGS. 9 and 10).

TABLE 4

| | | | | DPₐᵥ of | | | | |
|---|---|---|---|---|---|---|---|---|
| Enzymatic | MSer(OH)-glu conversion (%) | | | elongated | | | | |
| cascade | % | % | % | polymers | Type of osidic bonds (%) | | | |
| with GS-D | Total | Peak 1 | Peak 2 | (UV) | α-1,2 | α-1,3 | α-1,4 | α-1,6 |
| +GS-A | 70.3 | 23.2 | 96.5 | 23; 3 | — | — | — | 100 |
| +GS-B | 70.2 | 22.8 | 96.8 | 38 | N.D | N.D | N.D | N.D |
| +GS-C | 67.6 | 18.3 | 97 | 41 | N.D | N.D | N.D | N.D |
| +GS-F Δ1 | 77.7 | 45.9 | 96.3 | 9 | N.D | N.D | N.D | N.D |
| +GS-FS | 77.1 | 95.6 | 71.5 | 9 | — | 84 | — | 16 |
| +DSR-M | 80.2 | 58.7 | 95.7 | 37 | — | — | — | 100 |
| Δ1 | | | | | | | | |
| +DSR-M | 90.4 | 89.6 | 93.8 | 10 | N.D | N.D | N.D | N.D |
| Δ2 W624A | | | | | | | | |
| +DSR-G | 77.2 | 52.3 | 91.2 | 5 | N.D | N.D | N.D | N.D |
| Δ1 | | | | | | | | |
| +ASR Δ1 | 97.8 | 99 | 98.8 | 7 | — | 29 | — | 71 |
| +DSR-S | 90.48 | 87.1 | 95.9 | 19 | N.D | N.D | N.D | N.D |
| vardel Δ4N | | | | | | | | |
| +DSR-OK | 93.9 | 91.4 | 96.6 | 24; >24,000 | N.D | N.D | N.D | N.D |
| Δ1 | | | | | | | | |

Peak 1 = MSer(OH)-monoglucosylated;
Peak 2 = MSer(OH)-diglucosylated;
N.D.: not determined

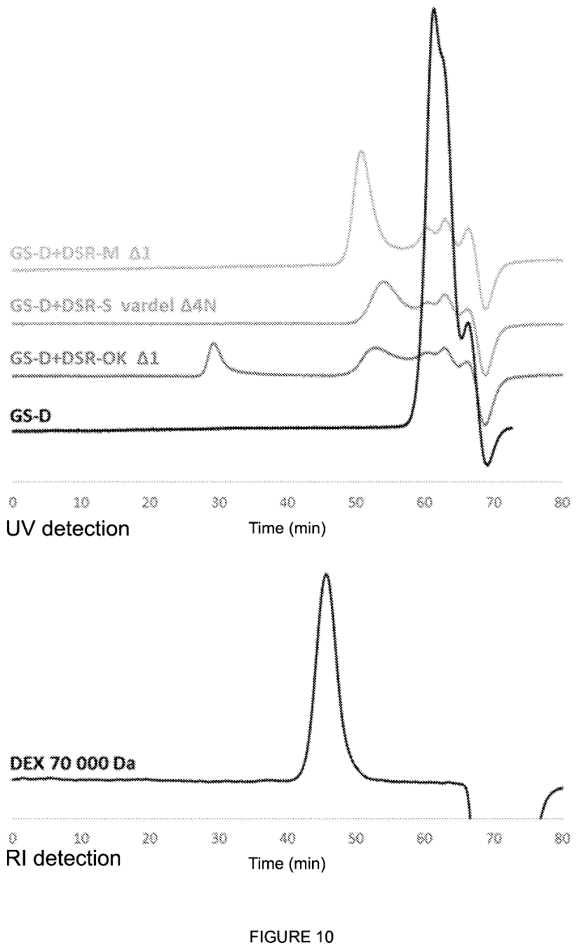

Notably, the DSR-M Δ1, DSR-M Δ2 and DSR-M Δ2 W624A enzymes very effectively extended MSer(OH)-glu, achieving MSer(OH)-glu conversion of 80.2% and 90.4% respectively. They produced a dextran-type motif, composed exclusively of glucosyl units linked by α-1,6 bonds (the natural motif of these enzymes), giving them a lower risk of anaphylactic shock. As shown in FIG. 11, it was possible to control polymer size by enzyme engineering, DSR-M Δ2 W624A being a variant of the DSR-M Δ1 enzyme [24]. The W624A mutation produced MSer(OH)-polym with a reduced average molar mass (10 units), whereas the reaction with the DSR-M Δ1 enzyme produced MSer(OH)-polym with a higher average molar mass, comprising an average of 37 glucosyl units.

Low molar mass dextrans are already marketed for pharmaceutical applications, notably in certain ophthalmic products. In free form, they are used as eye lubricants or artificial tears (see https://www.dextran.com/application-areas/eye). Their coupling to a MSer(OH) with high UV absorption capacity should allow the development of new products, formulations, (bio) materials and medicinal products combining the properties of sugars and mycosporines.

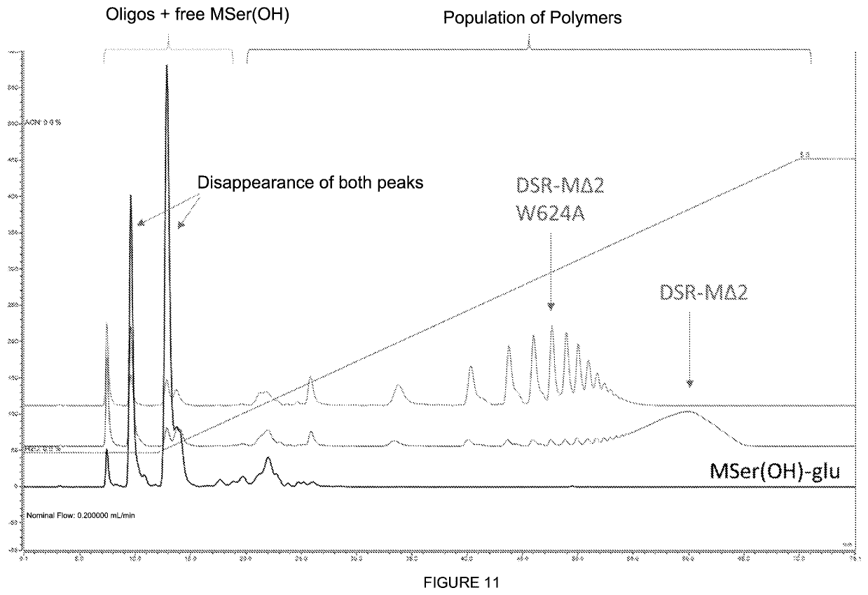

The absorption capacity of mycosporines was not affected by the grafting of a polysaccharide chain. Indeed, absorbance scans between λ=200 nm and λ=800 nm of MSer(OH)-polym products obtained with all of the enzymes tested, performed using a CARY 3 500 spectrophotometer showed that glucosylation and elongation of glucosyl chains on MSer(OH) did not cause any change in the maximum wavelength AMAX of absorbance (FIG. 12).

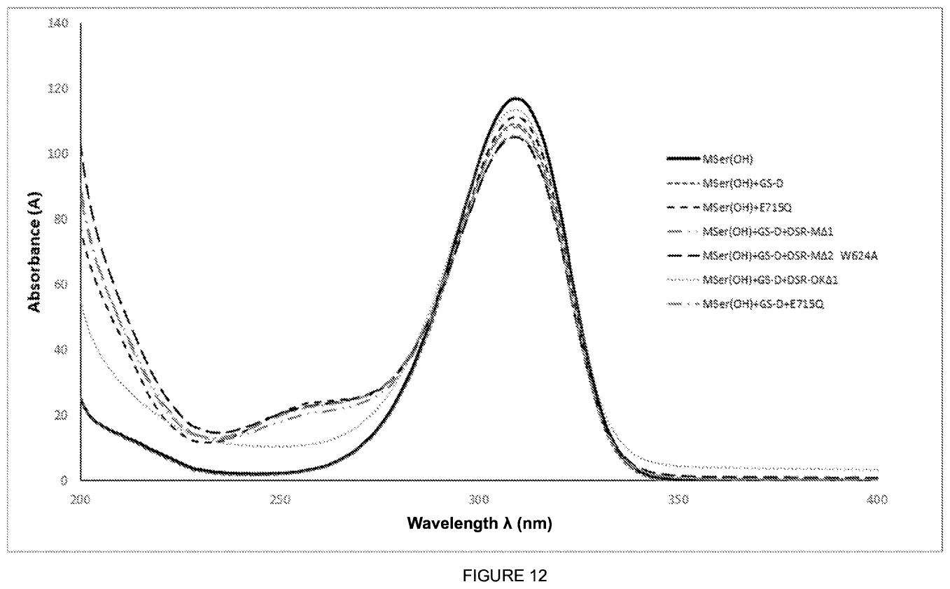

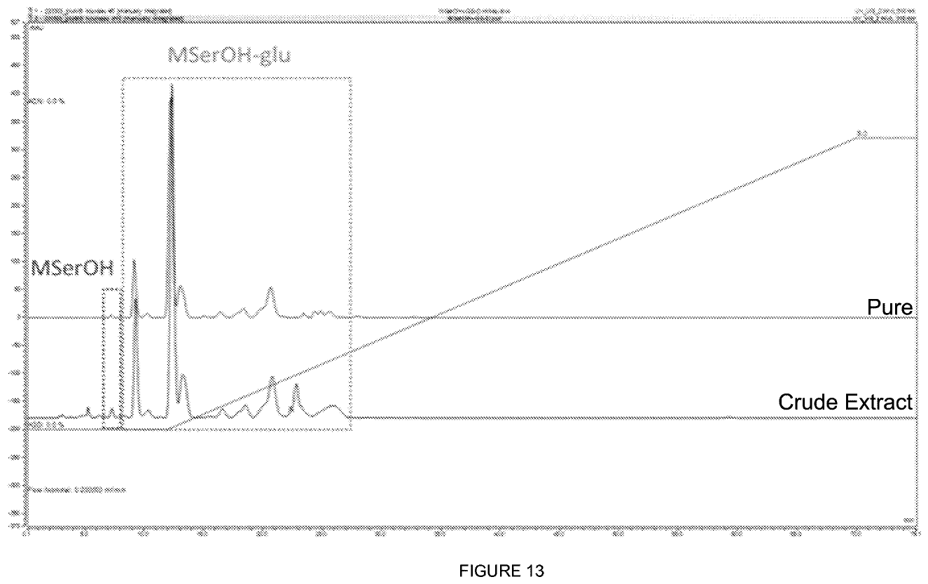

Finally, the glucosylation reaction was also tested on a more easily sourced crude extract of Lichina pygmaea, obtainable from an aqueous or hydroalcoholic extraction, optionally followed by lyophilization. The extract is derived directly from the lichen Lichina pygmaea, unpurified but enriched with MSer(OH) to 10% of the total extract mass. The first step of glucosylation by GS-D gave identical results to the reactions carried out on pure MSer(OH), that is, 95% glucosylated MSer(OH) (FIG. 13). The second step involving polymerases was also as efficient as when performed with purified MSer(OH) (FIG. 14 with the DSR-M Δ1 enzyme). Thus, the enzymes are not inhibited by other components of the crude medium. This is particularly advantageous for an industrial approach to the method, avoiding the costly MSer(OH) purification step.

Advantage of 2-Step Enzymatic Cascade Vs. One-Pot Synthesis

The use of GS-D and another polymerase in synergy (one-pot synthesis or co-synthesis for simultaneous glucosylation and extension of MSer(OH)) was evaluated and compared with the glucosylation yields obtained in two-step enzymatic cascades.

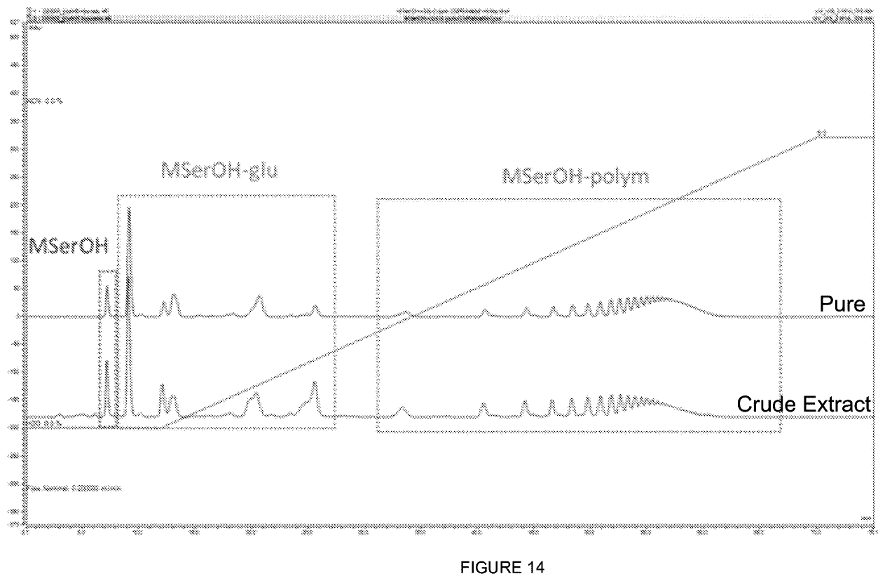

However, as shown by the comparison of FIG. 8 (enzymatic cascade) and FIG. 15 (co-synthesis), the overall MSer(OH) conversions obtained are less interesting in co-synthesis than in the enzymatic cascade. In co-synthesis, MSer(OH)-glu products are less consumed by polymerases, so there is a larger population of MSer(OH)-glu and a smaller population of MSer(OH)-polym.

### Example 2: Properties of MSer(OH)-Glu and MSer(OH)-Polym

The antioxidant properties and photostability of the MSer(OH); MSer(OH)-glu and MSer(OH)-polym products generated by the method of the invention were tested on reaction media purified from the DNA present in the cell extracts used for MSer(OH) glucosylation, in order to avoid DNA interference in the phenomena studied. This purification was carried out by anion exchange chromatography, using a Diaion resin.

### Photostability of Synthesized Products

Photostability was assessed on the various MSer(OH) glucosylation products. To achieve this, the samples were irradiated in a Sun Test XLS solar simulator (Atlas Material Testing Solutions, USA). The sun filter was selected to reproduce conventional sun exposure. The concentration of remaining molecules was monitored by measuring absorbance at λ=310 nm. The photodegradation kinetics obtained can be used to calculate the photodegradation quantum yield of the products. This quantum yield represents the ratio between the photodegraded molecules and the number of photons received by these molecules, and is calculated according to the following formula:

$$Φ = \frac{{r}_{0} \cdot V \cdot {N}_{A}}{{I}_{0} ( 1 - {10}^{- ( A )} )}$$

where r₀ is the rate of photodecomposition (mol·L⁻¹·s⁻¹), V is the volume of solution (L), NA is the Avogadro number (mol⁻¹), I₀ is the number of photons emitted in the MSer(OH) absorbance band range and A is the absorbance of the solution of the different samples. Experiments were carried out in triplicate.

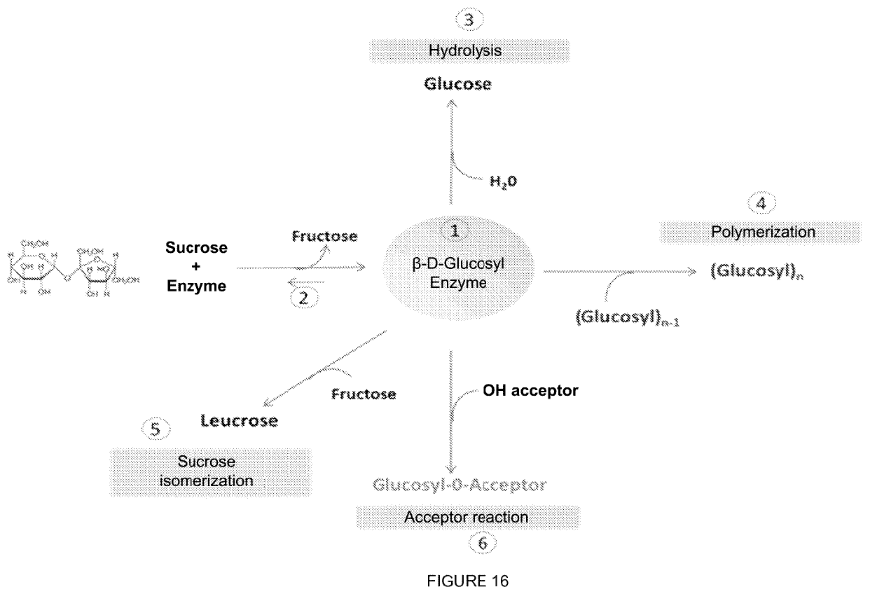

All MSer(OH) glucosylation and elongation products exhibit high photostability, with quantum yields of the order of 10⁻⁵ (FIG. 17), comparable to those of some sunscreens as well as other mycosporines [25, 26].

Glucosylation of mycosporine therefore has no impact on photostability. This is because the glucosylation positions do not prevent delocalization of the electron cloud, allowing the energy received to dissipate in the form of heat. Also, the length of the carbohydrate chain does not affect the photostability of the products.

However, preliminary tests have highlighted the role of DNA in the photostability of products, via the generation of reactive species. This translates into poorer photostability of the product from the MSer(OH)+GS-D+DSR-M Δ2 W624A cascade, whose DNA purification was the least efficient.

Finally, it appears that the product of the MSer(OH)+GS-D+DSR-OK Δ1 cascade has greater photostability than free MSer(OH). This can be explained by the physical protection provided by the long dextran chain, which protects the MSer(OH) from irradiation.

### Antioxidant Power

The antioxidant power of MSer(OH)-glu and Mser(OH)-polym glucosylation products was determined according to the TEAC (Trolox Equivalent Antioxidant Capacity) method. The TEAC test is well established for assessing radical scavenging properties and allows TEAC scores to be compared with a wide bibliography. Based on the reduction of the previously oxidized ABTS+ form, this spectrophotometric method is easy to implement [27]. The reduction of ABTS+ results in a drop in absorbance at λ=734 nm and is proportional to the concentration of the antioxidant molecule. The TEAC score is established by comparing the director coefficients of % inhibition=f (antioxidant concentration) with those obtained with Trolox, a reference antioxidant molecule. Experiments were carried out in triplicate.

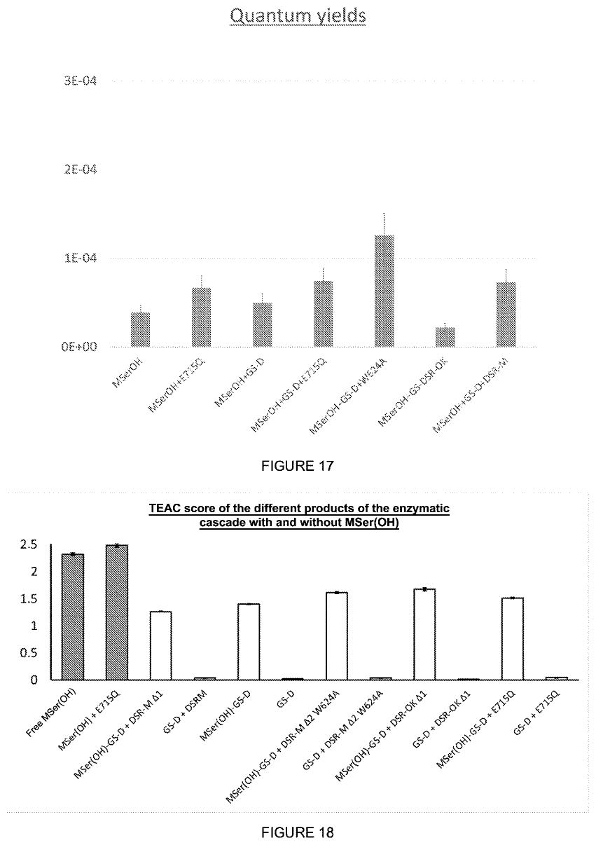

The scores obtained for MSer(OH) and the glucosylated derivatives thereof are all greater than 1 (FIG. 18). The products tested therefore display a strong antioxidant power and this power is retained for all the glucosylation products obtained, in contrast to what was observed when flavonoids such as luteolin were glucosylated [28]. The antioxidant power of products derived from MSer(OH)-free enzymatic cascades was assessed as virtually nil, attesting that this biological property is indeed due to the mycosporine portion of the molecule.

To check that the test was carried out correctly, we also checked the TEAC score of a well-known antioxidant, ascorbic acid or vitamin C. The value obtained (0.8 TEAC) is in line with those reported in the literature [29]. Thus, according to our test, all the products in the enzymatic cascade have greater antioxidant power than ascorbic acid.

### LIST OF REFERENCES

1. Lawrence, K. P., Long, P. F. & Young, A. R. Mycosporine-Like Amino Acids for Skin Photoprotection. Curr. Med. Chem. 25, 5512-5527 (2018)
2. de la Coba, F. et al. UVA and UVB Photoprotective Capabilities of Topical Formulations Containing Mycosporine-like Amino Acids (MAAs) through Different Biological Effective Protection Factors (BEPFs). Mar. Drugs 17 (2019)
3. Downs, C. A., DiNardo, J. C., Stien, D., Rodrigues, A. M. S. & Lebaron, P. Benzophenone Accumulates over Time from the Degradation of Octocrylene in Commercial Sunscreen Products. Chem. Res. Toxicol. 34, 1046-1054 (2021)
4. Vuckovic, D. et al. Conversion of oxybenzone sunscreen to phototoxic glucoside conjugates by sea anemones and corals. Science 376, 644-648 (2022)
5. Chrapusta, E. et al. Mycosporine-Like Amino Acids: Potential Health and Beauty Ingredients. Mar. Drugs 15 (2017)
6. Woolley, J. M. et al. Unravelling the Photoprotection Properties of Mycosporine Amino Acid Motifs. J. Phys. Chem. Lett. 9, 3043-3048 (2018)
7. Carreto, J. I. & Carignan, M. O. Mycosporine-Like Amino Acids: Relevant Secondary Metabolites. Chemical and Ecological Aspects. Mar. Drugs 9, 387-446 (2011)
8. Claverie, M., McReynolds, C., Petitpas, A., Thomas, M. & Fernandes, S. C. M. Marine-Derived Polymeric Materials and Biomimetics: An Overview. Polymers 12, 1002 (2020)
9. La Barre, S., Roullier, C. & Boustie, J. Mycosporine-Like Amino Acids (MAAs) in Biological Photosystems in Outstanding Marine Molecules: Chemistry, Biology, Analysis (ed. Stéphane La Barre, J.-M. K. (eds.)) 333-360 (Wiley-CH Verlag GmbH, 2014). doi: 10.1002/9783527681501.ch15
10. Shick, J. M. & Dunlap, W. C. Mycosporine-like amino acids and related Gadusols: biosynthesis, accumulation, and UV-protective functions in aquatic organisms. Annu. Rev. Physiol. 64, 223-262 (2002)
11. Böhm, G. A., Pfleiderer, W., Boger, P. & Scherer, S. Structure of a novel oligosaccharide-mycosporine-amino acid ultraviolet A/B sunscreen pigment from the terrestrial cyanobacterium Nostoc commune. J. Biol. Chem. 270, 853-8539 (1995)
12. Matsui, K. et al. Novel glycosylated mycosporine-like amino acids with radical scavenging activity from the cyanobacterium Nostoc commune. J. Photochem. Photobiol. B 105, 81-89 (2011)
13. Nazifi, E. et al. Characterization of the chemical diversity of glycosylated mycosporine-like amino acids in the terrestrial cyanobacterium Nostoc commune. J. Photochem. Photobiol. B 142, 154-168 (2015)
14. Ishihara, K. et al. Novel glycosylated mycosporine-like amino acid, 13-O-(β-galactosyl)-porphyra-334, from the edible cyanobacterium Nostoc sphaericum-protective activity on human keratinocytes from UV light. J. Photochem. Photobiol. B 172, 102-108 (2017)
15. CAZy-GH70. http://www.cazy.org/GH70.html
16. Molina, M., Cioci, G., Moulis, C., Séverac, E. & Remaud-Siméon, M. Bacterial α-Glucan and Branching Sucrases from GH70 Family: Discovery, Structure-Function Relationship Studies and Engineering. Microorganisms 9, 1607 (2021)
17. Vuillemin, M. et al. Characterization of the First α-(1-3) Branching Sucrases of the GH70 Family. J. Biol. Chem. 291, 7687-7702 (2016)
18. Malbert, Y. et al. Engineering a branching sucrase for flavonoid glucoside diversification. Sci. Rep. 8, 15153 (2018)
19. Fernandes, S. C. M. et al. Exploiting Mycosporines as Natural Molecular Sunscreens for the Fabrication of UV-Absorbing Green Materials. ACS Appl. Mater. Interfaces 7, 16558-16564 (2015)
20. Dunlap, W. C., Williams, D. M., Chalker, B. E. & Banaszak, A. T. Biochemical photoadaptation in vision: U.V.-absorbing pigments in fish eye tissues. Comp. Biochem. Physiol. Part B Comp. Biochem. 93, 601 (1989)
21. Wada, N., Sakamoto, T. & Matsugo, S. Mycosporine-Like Amino Acids and Their Derivatives as Natural Antioxidants. Antioxidants 4, 603-646 (2015)
22. Studier, F. W. Protein production by auto-induction in high density shaking cultures. Protein Expr. Purif. 41, 207-234 (2005)
23. Miller, G. L. Use of Dinitrosalicylic Acid Reagent for Determination of Reducing Sugar. Anal. Chem. 31, 426-428 (1959)
24. Claverie, M. et al. Futile Encounter Engineering of the DSR-M Dextransucrase Modifies the Resulting Polymer Length. Biochemistry 58, 2853-2859 (2019)
25. Jones, A. E., “A spectroscopic study of sunscreens”. Doctoral, Durham University. http://etheses.dur.ac.uk/4261/26.
26. Conde, F. R. et al. The Photoprotector Mechanism of Mycosporine-like Amino Acids. Excited-State Properties and Photostability of Porphyra-334 in Aqueous Solution. Journal of Photochemistry and Photobiology B: Biology 56 (2-3), 139-44 (2000)
27. P. Noury. Microplate assay of total antioxidant status using the TEAC method. 2016, pp. 12. hal-02602438v2
28. Malbert, Y., et al. Extending the Structural Diversity of α-Flavonoid Glycosides with Engineered Glucansucrases. ChemCatChem 6 (8), 2282-91 (2014)
29. Mira, L. M., et al. Measurement of Relative Antioxidant Activity of Compounds: A Methodological Note. Redox Report: Communications in Free Radical Research 4 (1-2): 69-74 (1999)

1. A method for producing a glucosylated mycosporine, said method comprising a step a) of glucosylating a mycosporine by the enzyme α-transglucosylase GS-D from sucrose to obtain a glucosylation intermediate which is a glucosylated mycosporine having an average degree of polymerization ranging from 1 to 2 glucosyl units.

2. The method according to claim 1 further comprising a step b) of elongating the carbohydrate portion of the glucosylation intermediate obtained in step a) by at least one α-transglucosylase GH-70 different from α-transglucosylase GS-D from sucrose, to obtain a glucosylated mycosporine having an average degree of polymerization ranging from 3 to 1,000,000 glucosyl units.

3. The method according to claim 2, wherein steps a) and b) are carried out concomitantly and comprise glucosylation of a mycosporine by the enzyme α-transglucosylase GS-D and at least one α-transglucosylase GH-70 different from α-transglucosylase GS-D from sucrose, to obtain a glucosylated mycosporine having an average degree of polymerization ranging from 3 to 1,000,000 glucosyl units.

4. The method according to claim 2, wherein said at least one α-transglucosylase of step b) is selected from the group consisting of: glucansucrases GS-A, GS-B, GS-C, GS-F Δ1 and GS-FS, dextransucrases DSR-M Δ1, DSR-M Δ2 W624A, DSR-G Δ1, DSR-S vardel Δ4N, and DSR-OK Δ1, alternansucrase ASR Δ1.

5. The method according to claim 1, wherein the mycosporine of step a) is selected from the group consisting of: pure mycosporine, mycosporine-enriched Lichina pygmaea lichen extract of 0 to 10% by extract weight.

6. The method according to claim 5, wherein the mycosporine is mycosporine-serinol (MSer(OH)).

7. A glucosylated mycosporine obtained by a method according to claim 1, said glucosylated mycosporine having an average degree of polymerization ranging from 1 to 2 glucosyl units.

8. The glucosylated mycosporine obtained according to claim 2, said glucosylated mycosporine having an average degree of polymerization ranging from 3 to 1,000,000 glucosyl units, preferably from 3 to 800,000 glucosyl units.

9. A cosmetic composition comprising by way of active ingredient a glucosylated mycosporine as defined in claim 7.

10. The glucosylated mycosporine according to claim 7, for use as a medicinal product.

11. The glucosylated mycosporine according to claim 7, for use in the prevention or treatment of cataract, albinism, xeroderma pigmentosum, skin cancer.
